(19) United States
(12) Patent Application Publication
(10) Pub. No.: US 2018/0051244 A1
(43) Pub. Date: Feb. 22, 2018

(54) USE OF GLASS BEADS AS A DRY THERMAL EQUILIBRATION MEDIUM

(71) Applicant: Diversified Biotech, Inc., Dedham, MA (US)

(72) Inventor: Daniel Perlman, Arlington, MA (US)

(21) Appl. No.: 15/238,940

(22) Filed: Aug. 17, 2016

(51) Int. Cl. C12M 1/02; C12N 1/00; F28D 20/00

(52) U.S. Cl. CPC C12M 41/22; F28D 20/0056; C12N 1/00

(57) ABSTRACT

System and method for controlling the temperature of a specimen by dry thermal equilibration using glass beads are provided. The method includes the use of a bed of glass beads that can be maintained at a relatively constant temperature in the range from about −80° C. to about +100° C. The system includes a container including the bed of glass beads and a temperature control mechanism capable of maintaining the temperature of the glass beads at a desired set temperature.

### BACKGROUND

Thermal control devices such as water baths and dry blocks are essential laboratory tools for heating, cooling or maintaining the temperature of laboratory vessels and the samples contained therein. Since these devices are often set at temperatures ideal for biological activity, they allow the growth of contaminating microorganisms, placing laboratory personnel at risk, compromising laboratory supplies and equipment, jeopardizing sterile operations, and requiring substantial routine instrument cleaning and maintenance. Furthermore, objects or vessels containing samples that are placed into the water of the laboratory thermal bath are prone to tipping over and floating. Such events can lead to the contamination or destruction of costly samples or sample contamination of the thermal bath and the laboratory. Moreover, thermal baths require frequent water replenishment and routine cleaning and maintenance, which can be time-consuming and costly.

As an alternative, dry solid thermal surfaces as well as particulate thermal media have been employed, as they reduce risks associated with water but have several additional drawbacks. For example, solid aluminum block systems limit the vessels that can be used to the size and shape of the drilled-out receptacles in their bodies. Due to their unique size or shape, laboratory vessels usually necessitate the purchase of numerous aluminum blocks or the costly production of custom aluminum block systems.

The use of particulate dry thermal bath media circumvents these issues, but such particulate media have additional limitations. They include problems in minimizing microbial contamination of the bath and challenges in physically supporting incubated objects in a stable position, while also providing effective thermal transfer properties.

One example of such particulate dry medium that has been employed in laboratories is the sand bath. Such sand baths are difficult and awkward to use for a variety of reasons. The shortcomings of sand baths include their accumulation of chemical contaminants and difficulty of cleaning/washing sand, the inconvenience of sand being adhesive and subject to static electric charge causing it to cling to lab containers, and the difficulty of physically inserting lab containers, e.g., beakers and flasks, into a bed of sand. Consequently, the use of sand baths is limited to heating samples that need to reach higher temperatures than water or oil baths can achieve.

As a high temperature sterilization medium, clean sand and glass beads have been used for accelerated heating (between 250 and 400° C.) of tools and instruments. However, the Food and Drug Administration (FDA states that sterilizing glass beads display “inconsistent heating and significant temperature variation” and are therefore not approved for sterilization procedures without premarket approval by the FDA, which has not been granted to date. As a likely consequence, glass beads have not been used in thermal equilibration devices such as laboratory incubation baths. Therefore, more recently, as an alternative thermal incubation medium, aluminum pellets have been introduced in thermal equilibration baths. However, widespread application of aluminum pellets is difficult because they are difficult to wash and clean; aluminum pellets are chemically reactive in acid and will corrode if autoclaved. They are also irregular in shape and require mechanical polishing, thereby increasing manufacturing costs and damage to glass vessels, and making it difficult to stabilize vessels in an aluminum pellet thermal medium.

Thus, there is a need for safe, effective, and easy to clean thermal media for controlling the temperature of laboratory specimens.

### SUMMARY OF THE INVENTION

The present invention provides a system for controlling the temperature of a specimen using glass beads as a dry incubation medium. One aspect of the invention is a method for controlling the temperature of a specimen. The method includes the steps of: (a) providing a container or a thermal control device including a plurality of glass beads, wherein the beads are essentially spherical and have a thermal conductivity from about 0.7 W/mK to about 1.9 W/mK, and wherein the glass beads are equilibrated at a selected temperature in the range from about minus 80° C. to about +100° C.; and (b) placing the specimen in contact with the glass beads, whereby the temperature of the specimen equilibrates with the temperature of the glass beads.

Another aspect of the invention is a system for controlling the temperature of a specimen by dry thermal equilibration. The system includes a container including a bed of glass beads for use as a dry thermal equilibration medium, the container configured for accommodating the specimen within the bed of glass beads; and a temperature control mechanism capable of maintaining the temperature of the bed of glass beads at a desired set temperature. Preferably, the system is not capable of sterilizing the specimen through the application of heat (i.e., it is not capable of heating the specimen to greater than 100° C. for a period of time sufficient for sterilization).

### BRIEF DESCRIPTION OF THE DRAWINGS

FIG. 1 is a schematic cross-sectional representation of an embodiment of a thermal system (1) for controlling the temperature of a sample in a vessel (2). The system includes a container (3) and a thermal equilibration medium consisting essentially of glass beads (4). The system also includes a thermal control device, which includes a container vessel (5) and a power source (6). Also depicted are optional thermal source (7), optional temperature control unit (8), and optional thermal insulation (9).

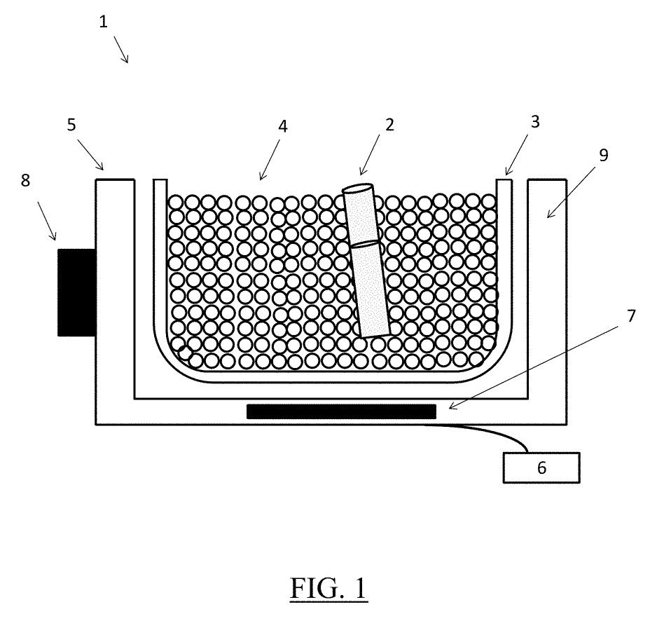

### DETAILED DESCRIPTION OF THE INVENTION

The present invention provides a system and method for controlling the temperature of a specimen using glass beads as a dry incubation medium having optimal shape and size, as well as maintenance and contamination control benefits.

One aspect of the invention is a method for controlling the temperature of a specimen. The method includes the steps of: (a) providing a container or a thermal control device including a plurality of glass beads, wherein the beads are essentially spherical and have a thermal conductivity from about 0.7 W/mK to about 1.9 W/mK, and wherein the glass beads are equilibrated at a selected temperature in the range from about −80° C. (the approximate temperature of dry ice) to about +100° C. (the temperature of boiling water); and (b) placing the specimen in contact with the glass beads, whereby the temperature of the specimen equilibrates with the temperature of the glass beads. In an embodiment of the method, the specimen is not sterilized by carrying out the method; sterilization requires heating of a device or specimen to a temperature greater than 100° C. for a period of time sufficient to kill essentially all microorganisms. Sterilization procedures and devices do not utilize incubation and equilibration of experimental samples at controlled temperatures as does the present invention.

In some embodiments, the method includes several hundred to tens of thousands of essentially spherical glass beads, which can be pre-equilibrated to a desired incubation temperature from about −80° C. to about +100° C. and placed in a container. In some embodiments, the container is any type of suitable vessel. For example, the vessel may be a polyethylene, polypropylene or polystyrene thermoplastic vessel that may be injection-molded or blow-molded, a glass bottle or glass laboratory vessel or a metal vessel (such as an aluminum vessel), or an insulated vessel. In certain embodiments, the glass beads, initially at room temperature, can be placed in a thermal control device, such as an active heating device or a cooling device and brought to a desired equilibration temperature. The method may further include glass beads being then placed in a container or in an active thermal control device. A laboratory specimen to be thermally equilibrated may then be placed in direct contact with the glass beads (e. g., submerged or partially submerged in the beads), whereby the temperature of the specimen equilibrate with the temperature of the glass beads.

In some embodiments, the glass beads have diameters from about 2 mm to about 10 mm. In an embodiment, the glass beads have a diameter from about 2 mm to about 3 mm or from about 2 mm to about 4 mm. In other embodiments, the glass beads have a diameter from about 3 mm to about 5 mm. Glass beads as manufactured from molten glass are essentially smooth and spherical, easily rolled and rotated, thereby allowing easy insertion and removal of any object from a bed of such glass beads. They are glassy smooth without any need for polishing before use. Eliminating the polishing step saves considerably on manufacturing costs, and its smooth surface does not present risk of scratching or damaging glass labware.

In some embodiments, glass beads are made from common soda lime glass, borosilicate glass, or any other conventional silica-based glass material to form a dry, temperature-equilibrating bed of glass beads. In certain embodiments, the glass beads have a thermal conductivity from about 0.7 W/mK to about 1.9 W/mK. In some embodiments, the glass beads have a thermal conductivity from about 1.1 W/mK to about 1.3 W/mK. The materials of the beads are dry and naturally more resistant to microbial growth than water and therefore less likely to harbor or support the viability of microbes or transmit microbes in the laboratory. A further advantage of the glass beads over other dry thermal particulate media, such as aluminum pellets and sand baths is that glass beads are impermeable and chemically inert and are easily washed (even acid washed), dried and/or sterilized by autoclaving or baking. By comparison, aluminum pellets are chemically reactive in acid, more irregular in shape, more difficult to wash and clean, and will corrode if autoclaved, while sand baths accumulate chemical contaminants and are extremely difficult to clean.

Sand particles are defined herein as ranging in diameter from approximately 0.1 mm up to a maximum diameter of under 2 mm, i.e., 1.9 mm or less. Sands are fragmented and usually irregular in appearance and may be created either by natural events, e.g., by erosion or freeze-thaw cycles, or artificially by mechanically crushing larger rocks and stones. The mineral composition of sands varies widely and includes relatively pure silicon dioxide-based sands used in the manufacture of common glass. Glass beads of the present invention are physically distinct from sands and are defined by their being man-made from molten glass containing primarily silicon dioxide, and by a process that results in essentially spherical particles that are physically distinct from fragmented, randomly shaped sand particles. Furthermore, to be useful in the present invention, glass beads are larger in diameter than sand particles and are at least 2.0 mm in diameter and preferably larger (e.g., 2.1 mm or larger and preferably 2.2 mm or larger, or 2.2-2.5 mm, 2.2-3.0 mm, 2.5-3.5 mm, 2.5-4 mm, 2.5-5 mm, 3.0-4 mm, 3.0-4.5 mm, 3.5-5 mm) based on their weight average diameter. The inventor has surprisingly found that incubation of chilled test tubes or specimens in beds of glass beads smaller in diameter than 2 mm often results in glass beads undesirably clinging to the outside of the tubes and specimens upon their removal from the beads. It is believed that either static electricity or ambient moisture condensation or both of these environmental factors on the outside surfaces of these objects, combined with the small diameter/light weight of the glass beads enables this cling.

In some embodiments, the ratio between the volume of glass beads and the volume of the specimen is from about 2 to about 100.

In certain embodiments, the thermal control device is a laboratory water bath or dry thermal bath including a container for a thermal equilibration medium and a heat source. In some embodiments, the specimen is maintained at a relatively constant temperature in the range from about −80° C. to about +100° C. In certain embodiments, the specimen is maintained within ±1° C., or ±2° C., or ±3° C., or ±4° C. of the desired temperature. In some embodiments, the laboratory specimen to be equilibrated is a thermoplastic or glass or metal laboratory vessel containing a liquid, solid or gas sample.

Another aspect of the invention is a system for controlling the temperature of a specimen by dry thermal equilibration. The system includes a container including a bed of glass beads for use as a dry thermal equilibration medium, the container configured for accommodating the specimen within the bed of glass beads; and a temperature control mechanism capable of maintaining the temperature of the bed of glass beads at a desired set temperature.

In some embodiments the thermal equilibration medium is positioned in the container in a manner such that the specimens can be inserted within the medium in thermal communication with the medium.

In some embodiments, the system includes glass beads having diameters from about 2 mm to about 10 mm. In other embodiments, the glass beads have a diameter from about 2 mm to about 3 mm or from about 3 mm to about 4 mm or from about 3 mm to about 5 mm. In still other embodiments, the glass beads have a diameter from about 4 mm to about 6 mm. In some embodiments, the system includes glass beads made from common soda lime glass or borosilicate glass. In certain embodiments, the glass beads have a thermal conductivity from about 0.7 W/mK to about 1.9 W/mK. In some embodiments, the glass beads have a thermal conductivity from about 1.1 W/mK to about 1.3 W/mK. In some embodiments, the container is any type of suitable vessel. For example, the vessel may be a polyethylene, polypropylene or polystyrene thermoplastic vessel that may be injection-molded or blow-molded, a glass bottle or glass laboratory vessel or a metal vessel (such as an aluminum vessel), or an insulated vessel.

In some embodiments, the ratio between the volume of the glass beads and the volume of the specimen is from about 2 to about 100. In certain embodiments, the thermal control device is a laboratory water or dry bath including a container for a thermal equilibration medium and a heat source.

In some embodiments, the specimen is maintained at a relatively constant temperature in the range from about −80° C. to about +100° C. In certain embodiments, the specimen is maintained within ±1° C., or ±2° C., or ±3° C., or ±4° C. of the desired temperature. In some embodiments, the specimen to be thermally equilibrated is a laboratory vessel containing a liquid, solid, or gas sample.

### EXAMPLE 1

### Rates of Cooling using Different Cooling Media

A small amount of distilled water (3 ml sample) at 75° F. was placed inside a clinical centrifuge tube (Corning brand 15 ml capacity polypropylene tube). The amount of time required for the water sample in this centrifuge tube to be cooled to 45° F. when the tube was surrounded by different cooling media (pre-equilibrated to 32° F., held in an insulating polystyrene container) was measured using a stopwatch and a low mass thermocouple temperature probe to measure instantaneous temperature (ThermoWorks Inc. American Fork, Utah). Glass beads (1.2 mm and 2 mm diameter) consisted of soda lime glass and were obtained from Ceroglass Technologies, Inc., Columbia, Tenn. Aluminum pellets were also tested (LAB ARMOR BEADS) that are polished irregular-shaped rounded pellets approximately 5 mm in diameter, obtained from ThermoFisher Scientific, Inc.). Results (times required to cool the water sample from 75° F. to 45° F. in triplicate trials) were as follows:

| Cooling Medium | Time (min) |
|---|---|
| wet water-ice | 2.0-2.5 |
| dry 2 mm glass beads | 4.0-4.5 |
| dry 1.2 mm glass beads | 4.5-5.0 |
| dry 5 mm aluminum pellets | 4.0-4.5 |

Cooling rates for 3 ml water held in a polypropylene centrifuge tube were comparable for all cooling media except for wet water-ice that cooled the water (decreasing 30° F.) in approximately half the time required for the dry thermal media. Comparing the dry beads and pellets, it is remarkable that the aluminum pellet medium (aluminum having a 200-fold greater thermal conductivity than glass) failed to cool the centrifuge tube's water sample any faster than did the glass beads. While not wishing to be limited or bound by theory, it is possible that what limits and determines the rate of thermal transfer and cooling of the water in the plastic centrifuge tube is not the thermal transfer medium surrounding the centrifuge tube but rather the polypropylene wall of the tube itself. In the present example, polypropylene used in molding the tube has a poor thermal conductivity (conductivity units being watts per meter degree). Polypropylene has a thermal conductivity approximately 5-8 fold lower than glass (0.1-0.2 for polypropylene versus approximately 1.0 for glass versus approximately 200 for aluminum. On the other hand, the heat capacities for glass and aluminum (energy required to raise the temperature of a material one degree C.) are very similar (0.90 Joules per gram-degree for aluminum versus 0.84 for soda lime glass).

### EXAMPLE 2

### Rates of Cooling by Glass Beads vs. Aluminum Pellets as Cooling Medium

A small volume of distilled water (3 ml) initially at 74° F. was placed inside a disposable 13 mm×100 mm glass test tube (Fisher Scientific). The rate of cooling of the water in the test tube surrounded by approximately 250 g of either of two different thermal equilibration media was followed and recorded over time. The two different thermal media (each pre-equilibrated to 32° F.) were as follows: (a) Dry 2.6 mm diameter soda lime glass beads (obtained from Ceroglass Technologies, Inc., Columbia, Tenn.), and (b) Dry Lab Armor aluminum pellets described in Example 1 above.

The pre-chilled thermal media were held in an insulating STYROFOAM container to minimize warming of the media over the course of the experiments. A low mass thermocouple probe (see Example 1) was used to measure instantaneous temperature. Results (time averages) from duplicate trials were as follows:

Water Temperature (° F.)

| Time (min) | Chilled Aluminum Pellets | Chilled Glass Beads |
|---|---|---|
| 0 | 74 | 74 |
| 0.5 | 67 | 67 |
| 1 | 59 | 60 |
| 1.5 | 54 | 56 |
| 2 | 50 | 53 |
| 2.5 | 46 | 52 |
| 3 | 45 | 50 |
| 3.5 | 43 | 48 |
| 4 | 41 | 47 |
| 4.5 | 40 | 46 |
| 5 | 40 | 46 |
| 6 | 39 | 45 |
| 7 | 39 | 44 |

The cooling rate for 3 ml water contained in a glass test tube was somewhat faster using a dry aluminum pellet cooling medium compared to a dry glass bead cooling medium. Comparing the rate of cooling of the water sample using the external glass bead medium and the external aluminum pellet medium, it is remarkable that the aluminum pellets only modestly out-performed the glass beads given that aluminum has a 200-fold greater thermal conductivity than glass.

While not wishing to be limited or bound by any theory, it is considered likely that what limits the rate of thermal transfer and cooling of the water in the glass test tube is not principally the thermal conductivity of the thermal transfer medium surrounding the glass tube but rather the glass wall of the tube itself. In the present example, the thermal conductivity (watts per meter degree) for glass is approximately 1.0 versus approximately 200 for aluminum. Therefore, it is proposed that while chilled aluminum pellets should theoretically remove heat much more rapidly than chilled glass beads when directly contacting a warm surface, in the present example the aluminum pellets are separated from the warm water in the test tube by the tube's surface. Furthermore the aluminum pellets establish only very limited contact with the glass test tube's surface owing to the irregular shapes of the pellets. By comparison, it is likely that the smaller and regularly shaped chilled glass beads actually establish better physical contact than the aluminum pellets with the glass test tube's surface. Better physical proximity between the beads and the test tube surface may offset the greater thermal conductivity of the aluminum pellets because contact between these pellets and the glass test tube surface is relatively poor. Therefore, the difference in the rate of cooling for dry aluminum pellets versus dry glass beads in this example is surprisingly small considering the 200-fold difference in their thermal conductivities.

1. A system for controlling the temperature of a specimen by dry thermal equilibration, the system comprising:
a container comprising a bed of glass beads, the glass beads having a diameter of about 2 mm to about 10 mm, for use as a dry thermal equilibration medium, the container configured for accommodating the specimen within the bed of glass beads; and
a temperature control mechanism capable of maintaining the temperature of said bed of glass beads at a desired set temperature.

2. (canceled)

3. The system of claim 1, wherein the glass beads comprise soda lime glass or borosilicate glass.

4. The system of claim 1, wherein the glass beads have a thermal conductivity from about 0.7 W/mK to about 1.9 W/mK.

5. The system of claim 1, further comprising said specimen disposed within the bed of glass beads, wherein the volume ratio of glass beads to specimen is from about 2 to about 100.

6. The system of claim 1, wherein the system comprises a temperature-regulating laboratory incubation device.

7. The system of claim 6, wherein the laboratory incubation device is top-loaded with a dry thermal equilibration medium and at least one specimen.

8. The system of claim 1 that is capable of maintaining the specimen at said desired temperature within ±2° C.

9. The system of claim 1, wherein the system is capable of maintaining said temperature over a range from about −80° C. to about +100° C.

10. The system of claim 1 that is not capable of sterilizing the specimen through the application of heat.

11. The system of claim 1, wherein the specimen is a laboratory vessel containing a liquid, solid, or gas sample.

12. A method for controlling the temperature of a specimen, the method comprising the steps of:
(a) providing a container or a thermal control device comprising a plurality of glass beads, wherein the beads have a diameter of about 2 mm to about 10 mm, are essentially spherical and have a thermal conductivity from about 0.7 W/mK to about 1.9 W/mK, and wherein the glass beads are equilibrated at a selected temperature in the range from about −80° C. to about +100° C.;
(b) placing the specimen in contact with the glass beads, whereby the temperature of the specimen equilibrates with the temperature of the glass beads.

13. (canceled)

14. The method of claim 12, wherein the glass beads comprise soda lime glass or borosilicate glass.

15. The method of claim 12, wherein the glass beads have a thermal conductivity from about 1.1 W/mK to about 1.3 W/mK.

16. The method of claim 12, wherein the volume ratio of glass beads to specimen is from 2 to about 100.

17. The method of claim 12, wherein the thermal control device is a laboratory water bath apparatus free of water, or the thermal control device is another temperature-regulating laboratory incubation device.

18. The method of claim 12, wherein the specimen is maintained at said selected temperature within ±2° C.

19. The method of claim 12, wherein the specimen is a laboratory vessel containing a liquid, solid, or gas sample.

20. The method of claim 12, wherein the specimen is not sterilized by the method.

21. The system of claim 1, further comprising a specimen disposed within the bed of glass beads.
